(19) United States
(12) Patent Application Publication
(10) Pub. No.: US 2004/0072911 A1
(43) Pub. Date: Apr. 15, 2004

(54) Composition for treating hemorrhoids

(76) Inventor: L. Gaye Carroll, Magnolia, AR (US)

Correspondence Address: R. Keith Harrison, 2139 E. Bert Kouns, Shreveport, LA, 71105, US

(21) Appl. No.: 10/190,718

(22) Filed: Oct. 10, 2002

(51) Int. Cl. A61K 31/135; A61K 9/14

(52) U.S. Cl. 514/646

(57) ABSTRACT

A composition and method for the treatment of hemorrhoids. More particularly the present invention relates to a hemorrhoid-treating composition which includes a mixture of the antiseptic and bactericide gentian violet (methylrosaniline) and a lubricant such as petroleum jelly, also known as petrolatum or mineral jelly. The composition imparts a soothing and healing effect on swollen hemorrhoids, and particularly, on bleeding hemorrhoids.

### BACKGROUND OF THE INVENTION

1. Field of the Invention

The present invention relates to a composition which is particularly useful in the treatment of hemorrhoids. More particularly, the present invention relates to a hemorrhoid-treating composition which includes a mixture of the antiseptic and bactericide gentian violet (methylrosaniline) and a lubricant such as petroleum jelly, also known as petrolatum or mineral jelly. The composition imparts a soothing and healing effect on swollen hemorrhoids, and particularly, on bleeding hemorrhoids.

Hemorrhoids are swollen blood vessels in the rectum. There are two basic types of hemorrhoids: internal and external. Internal hemorrhoids are swollen and inflamed veins far up in the rectum. Internal hemorrhoids cannot be seen or felt and usually are not painful due to the paucity in nerve endings in the upper portion of the rectum. While internal hemorrhoids are most commonly manifested by anal bleeding, they may prolapse, or protrude outside the anal sphincter. Usually, prolapsed internal hemorrhoids may be gently pushed back into place in the rectum.

External hemorrhoids are swollen blood vessels in the anus and are usually manifested by pain as well as bleeding. When external hemorrhoids prolapse, or protrude from the anal sphincter, blood clots sometimes form, causing an extremely painful condition known as thrombosis. While they usually disappear by themselves within about a week, thrombosed hemorrhoids may be removed by a physician or may be treated with a pain-reducing medication to reduce the pain.

It is believed that hemorrhoids are caused by the exertion of abdominal pressure on rectal veins, causing the veins to swell and become irritated. The abdominal pressure may be caused by a variety of factors and conditions including obesity, pregnancy, prolonged standing or sitting, liver disease, straining during bowel movements, coughing, sneezing, vomiting or holding the breath during physical activity. Hemorrhoids are largely preventable by the adoption of a high-fiber diet. On the other hand, persons whose diet consists largely of low-fiber, processed foods tend to run the highest risk of developing hemorrhoids. Furthermore, inadequate fluid intake can contribute to the development of hemorrhoids by causing the development of hard stools which irritate and inflame the rectal veins.

About half of persons living in the United States will be afflicted with hemorrhoids at some point during their lives. Hemorrhoids most often strike persons between the ages of 20 and 50. Some evidence indicates that “weak” veins, which are most susceptible to developing hemorrhoids, are inherited.

### SUMMARY OF THE INVENTION

The present invention is generally directed to a composition for the treatment of hemorrhoids. More particularly, the present invention relates to a hemorrhoid-treating composition which includes a mixture of the antiseptic and bactericide gentian violet (methylrosaniline) and a lubricant such as petroleum jelly, also known as petrolatum or mineral jelly. The composition imparts a soothing and healing effect on swollen hemorrhoids, and particularly, on bleeding hemorrhoids.

### DESCRIPTION OF THE PREFERRED EMBODIMENTS

The present invention includes a composition for the treatment of hemorrhoids, particularly external hemorrhoids. The composition is easy to apply and promotes pain reduction, as well as healing, of the hemorrhoids, including inflammation and torn tissue associated with hemorrhoids. In a preferred embodiment, the composition includes a mixture of non-prescription strength, 1% gentian violet (methylrosaniline) which is thoroughly mixed with a lubricant such as petroleum jelly, also known as petrolatum or mineral jelly. The methylrosaniline is an effective antiseptic and bactericide which prevents or fights infection and reduces pain. The lubricant provides a vehicle for effective delivery of the methylrosaniline to the inflamed hemorrhoid tissue. In a typical embodiment, the composition includes about seven (7) drops of the liquid methylrosaniline bactericide/antiseptic per teaspoon of the lubricant. The bactericide/antiseptic may be dispensed from a medicine dropper such as an EZY CARE (trademark) straight-tip or bent-tip, glass medicine dropper available from Apothecary Products, Inc. of Minneapolis, Minn. The EZY CARE (trademark) medicine dropper has a capacity of 1 ml of liquid, or approximately 30-35 drops. Accordingly, in a preferred embodiment the composition mixture includes at least about 0.2 ml to about 0.3 ml of the methylrosaniline per teaspoon of lubricant. However, it is understood that other types of medicine droppers may be used to dispense the drops into the lubricant. After the methylrosaniline is added to the lubricant, the two components are thoroughly mixed until the composition mixture assumes the substantially homogenous violet color of the methylrosaniline. After formation, the composition is typically placed in a typically 6 oz. capped, plastic applicator tube for subsequent application.

One possible method of applying the composition to the inflamed hemorrhoidal tissue includes dispensing the composition from a typically 6-ounce, capped plastic tube. A replaceable applicator cap having a smooth, rounded applicator tip may be provided on the capped tube for applying the composition to the hemorrhoids in the anal area. Preferably, the applicator tip is ¾″ long, and the top of the 6-ounce tube, as well as the base of the applicator tip, is ¼″ in diameter. The applicator tip tapers from the ¼″ base to the ⅛″ tip thereof. Applicator openings are provided in the sides of the applicator tip for dispensing the composition from the tip to the inflamed hemorrhoidal tissue.

It is understood that the tube and applicator cap heretofore described represent only one example of storage and application of the composition. Accordingly, the composition may be stored in any type of suitable container for subsequent application, and may be applied through the applicator cap heretofore described or through any other type of applicator or other instrument suitable for the purpose. The composition may also be applied manually to the inflamed area. Furthermore, the fingers of the patient afflicted with prolapsed hemorrhoids may lubricate his or her fingers with the composition mixture and use the lubricated fingers to gently push the prolapsed hemorrhoidal tissue back into the anal canal. The composition mixture reduces hemorrhoidal pain and promotes healing of the inflamed hemorrhoidal tissue.

The invention will be better understood by consideration of the following examples.

### EXAMPLE 1

A composition mixture was prepared by dropping seven (7) drops of non-prescription strength gentian violet (methylrosanaline) bactericide/antiseptic, using an EZY CARE (trademark) medicine dropper, into one (1) teaspoon of petroleum jelly and then thorougly mixing the methylrosanaline with the petroleum jelly until the resulting composition mixture assumed the substantially homogenous violet color of the methylrosanaline. The composition mixture was placed in 6-ounce capped tube having an applicator cap for subsequent application of the composition to inflamed hemorrhoidal tissue.

### EXAMPLE 2

The composition mixture prepared according to EXAMPLE 1 above was applied to the inflamed hemorrhoidal tissue of a patient using the applicator cap on the tube. The composition mixture was applied as often as necessary to relieve pain. The composition mixture significantly reduced pain associated with the hemorrhoids and promoted healing of the inflamed hemorrhoidal tissue.

### EXAMPLE 3

A patient afflicted with inflamed hemorrhoids applied the composition mixture prepared according to EXAMPLE 1 to the patient's inflamed hemorrhoidal tissue using the patient's fingers. The composition mixture was applied as often as necessary to relieve pain. The composition mixture significantly reduced pain associated with the hemorrhoids and promoted healing of the inflamed hemorrhoidal tissue.

### EXAMPLE 4

A patient afflicted with prolapsed hemorrhoids lubricated the patient's fingers using the composition mixture prepared according to EXAMPLE 1 above. The patient used the lubricated fingers to gently push the prolapsed hemorrhoids back into the patient's anal canal. The composition significantly reduced pain associated with the hemorrhoids and promoted healing of the inflamed hemorrhoidal tissue.

While the preferred embodiments of the invention have been described above, it will be recognized and understood that various modifications can be made in the invention and the appended claims are intended to cover all such modifications which may fall within the spirit and scope of the invention.

Having described my invention with the particularity set forth above, I claim:

1. A composition for treating hemorrhoids, comprising:
a quantity of lubricant; and
a quantity of methylrosanaline mixed with said quantity of lubricant to define a composition mixture.

2. The composition of claim 1 wherein said lubricant comprises petroleum jelly.

3. The composition of claim 1 wherein said methylrosanaline and said lubricant are present in said composition mixture in a ratio of at least about 0.2 ml to about 0.3 of said methylrosanaline per teaspoon of said lubricant.

4. The composition of claim 3 wherein said lubricant comprises petroleum jelly.

5. A composition for treating hemorrhoids prepared by mixing a quantity of methylrosanaline with a quantity of lubricant to define a composition mixture.

6. The composition of claim 5 wherein said lubricant comprises petroleum jelly.

7. The composition of claim 5 wherein said methylrosanaline and said lubricant are present in said composition mixture in a ratio of at least about 0.2 ml to about 0.3 ml of said methylrosanaline

8. The composition of claim 7 wherein said lubricant comprises petroleum jelly.

9. A method of forming a composition for treating hemorrhoids, comprising:
providing a quantity of lubricant;
adding a quantity of methylrosanaline to said quantity of lubricant; and
mixing said methylrosanaline with said lubricant to define a composition mixture.

10. The method of claim 9 wherein said lubricant comprises petroleum jelly.

11. The method of claim 9 wherein said methylrosanaline is present in said composition mixture in a ratio of at least about 0.2 ml to about 0.3 ml of said methylrosanaline per teaspoon of said lubricant.

12. The method of claim 11 wherein said lubricant comprises petroleum jelly.
